(19) United States
(12) Patent Application Publication
(10) Pub. No.: US 2014/0165365 A1
(43) Pub. Date: Jun. 19, 2014

(54) Apparatus and Method for Crimping an Implant

(71) Applicant: BIOTRONIK AG, Buelach (CH)

(72) Inventor: Amir Fargahi, Buelach (CH)

(73) Assignee: BIOTRONIK AG, Buelach (CH)

(21) Appl. No.: 14/093,792

(22) Filed: Dec. 2, 2013

Related U.S. Application Data
(60) Provisional application No. 61/736,555, filed on Dec. 13, 2012.

(51) Int. Cl. A61F 2/95

(52) U.S. Cl. CPC A61F 2/95
USPC 29/515; 29/283.5

(57) ABSTRACT

An apparatus and method for crimping an implant (30), in particular an intraluminal endoprosthesis, which can adopt either a compressed state or an expanded state, at least over part of its length including a roll arrangement comprising at least three rolls (1, 2, 3) for clamping the implant (30) therebetween, wherein the spacing of at least one roll (1) of the roll arrangement relative to each of the other at least two rolls (2, 3) can be reduced in such a way that an implant (30) clamped between the rolls (1, 2, 3) can be transferred from the expanded state into the compressed state, at least over part of its length.

### CROSS REFERENCE TO RELATED APPLICATIONS

This application claims benefit of priority to U.S. provisional patent application Ser. No. 61/735,555 filed Dec. 13, 2013.

### TECHNICAL FIELD

The present invention relates to an apparatus for crimping an implant, in particular an intraluminal endoprosthesis, and to a corresponding method.

### BACKGROUND

A wide variety of medical implants, in particular intraluminal endoprostheses, are known from the prior art for a wide range of applications. Within the context of the present invention, implants are to be understood to be endovascular prostheses or other endoprostheses, for example stents (vessel stents (vascular stents, including stents for application in the area of the heart and heart valve stents, for example mitral valve stents and pulmonary valve stents) and bile duct stents), endoprostheses for closing patent foramen ovale (PFO), stent grafts for treating aneurysms, endoprostheses for closing an ASD (atrial septal defect), and prostheses in the area of hard and soft tissue. An implant of this type is often inserted by means of a catheter into the organ or vessel to be treated.

In many cases, stents and other implants have an open-worked hollow cylindrical (tubular) and/or hollow conical main structure, which is open at both longitudinal ends, wherein the main structure is often formed of a multiplicity of crosspieces. In a main structure of this type, in the case of a heart valve stent for example, valvular cusps, for example three valvular cusps, which form the heart valve, may be arranged on the inner face and may consist of a plastic or a biological material, for example porcine pericardium. In this case the stent carries the heart valve and anchors it in the heart.

Stents and other implants normally adopt two states: namely a compressed state with a small diameter and an expanded state with a larger diameter. In the compressed state, the implant can be introduced by means of a catheter through narrow vessels into the vessel or organ to be treated and can be positioned at the point to be treated. To this end, the implant is crimped and is thus transferred from the expanded state of larger diameter into the compressed state of smaller diameter, at least over part of its length. The implant is then dilated at the treatment location, for example by means of the balloon of the catheter, and then adopts the expanded state again, in which the implant remains in the vessel or organ and is fixed there, once the catheter has been removed from the body of the patient being treated. Alternatively, if the main structure of an implant consists of a self-expanding material (for example Nitinol), said implant adopts the compressed state as a result of compression below the transition temperature and adopts the expanded state above the transition temperature.

A heart valve prosthesis and a deflecting device are known from document U.S. Pat. No. 8,029,564 B2. The system also includes a line, which is guided through the free ends of the stent posts, which carry the heart valve. The stent posts can be deflected inwardly by means of this line so as to achieve a closed state. This system is not suitable however for non-invasive transfer by means of a catheter to the point to be treated, since it is too bulky in the foot region.

A crimping tool is known from document US 2011/0056064 A1, which is very cost-intensive in terms of production since it is of complex design with a multiplicity of parts that are to be manufactured precisely and have to move synchronously. The crimping tool is formed in particular of a multiplicity of bars, which are arranged adjacently along the circumference of a circle and rotatably about an axis running transverse to the respective bar. The implant to be crimped can be received in the opening formed between the ends of the bar. Guide pins are displaced in a slot as a result of the movement of a lever, wherein a pin is arranged at a front end of each bar, and the radius of the opening formed between the bars is thus reduced or enlarged. For the crimping of a heart valve stent, the pins are displaced in such a way that the radius of the opening is decreased. With this tool, the stent is covered completely by said tool during the crimping process, since the bars and the pins are arranged between two plates and the tool therefore has a large expansion in the longitudinal direction of the stent and a comparatively high weight. For this reason the implant cannot be monitored visually during the crimping operation. Furthermore, it is not to be ruled out that the bars overlap as the opening radius is decreased, and there is thus a high risk of damage to the implant and to the tissue parts arranged against the implant. The large mass of the tool also means that it can only be brought below the transition temperature at great effort, which is necessary if the implant consists of a self-expanding material.

Document EP 2 229 921 A1 discloses an apparatus for crimping, which has a multiplicity of wires tensioned along a cylinder surface line between two spaced rings. If one of these rings is rotated relatively to the second ring about an axis parallel to these surface lines, the wires are thus twisted relative to the common axis so they form an hourglass-like geometrical area similarly to a rotational hyperboloid. The opening defined by the portion of the smallest diameter and formed between these wires is decreased or enlarged with increasing twisting of the rings. An implant can be arranged in this opening and can be crimped as the opening is decreased. This known apparatus is likewise of relatively complex design and is therefore difficult to assemble. The implant is covered during the crimping operation by the wires of the apparatus, such that the crimping process also cannot be monitored visually with this known apparatus. The risk of an overlap of segments of the implant during the crimping operation is high. It is also disadvantageous that the wires are tensioned more strongly at the two ends at which the wires are fastened to the opposite rings than in their middle region. This leads to a non-uniform distribution of the compression force onto the implant. Due to the many wires and the necessary large rings at the ends thereof, the known apparatus is relatively large and heavy.

### SUMMARY

The object is therefore to create an apparatus for crimping an implant of this type, said apparatus being less complex in design and being smaller and manageable. A method for crimping that can be carried out in a simple manner will be disclosed accordingly.

The above object is achieved by an apparatus having the features provided herein.

In particular, the apparatus according to the invention has a roll arrangement comprising at least three rolls for clamping the implant therebetween, wherein the spacing between at least one roll of the roll arrangement and each of the other at least two rolls can be reduced in such a way that an implant clamped between the rolls can be transferred from the expanded state into the compressed state, at least over part of its length.

Each roll of the roll arrangement is preferably fastened rotatably about its longitudinal axis in a C-shaped mount, wherein the longitudinal axes of the rolls preferably run parallel to one another and more preferably parallel to the longitudinal axis of the implant to be crimped, when the implant is clamped between the rolls.

The above-disclosed apparatus is relatively simple and small and allows external access to the implant to be crimped for monitoring purposes, since the C-shaped mount does not surround the implant completely. Furthermore, the arrangement of the implant between the at least three rolls has the advantage that the lateral surface of the preferably hollow cylindrical or hollow conical implant can roll over the lateral surfaces of the rolls. The risk of damage to the implant or to the tissue arranged against the main structure of the implant is thus considerably reduced. The housing of the apparatus, said housing accommodating the rolls and preferably being C-shaped, can be injection molded from plastic, thus achieving a further weight reduction and a further simplification in terms of handling. Standard components, which are very cost-effective, can be used as spindles, rolls and other components described below, which are used to reduce the spacing between the rolls. The compression force is additionally transferred to the implant by the rolls in a manner distributed homogeneously and uniformly over the entire length of the region to be crimped. Since plastic can be used for the housing, there are also advantages in terms of the weight of the apparatus such that the handling thereof is also facilitated.

The distance between the longitudinal axes of the rolls of the roll arrangement is defined as the spacing of said rolls relative to one another when the rolls are cut transverse to their longitudinal axis.

As already mentioned above, the rolls of the roll arrangement are preferably fastened in a C-shaped mount, which is particularly preferably formed in two parts. So as to be able to change the spacing between the rolls, the first part of the mount, to which merely one roll is preferably fastened, is arranged displaceably along a spindle fastened rotatably to the second part of the mount. The second part of the mount preferably has two rolls. The first mount part also has an inner thread portion, which cooperates with the outer thread of the spindle of the second mount part and engages therein. As the spindle rotates, the first mount part moves against the force of a compression spring mounted between the first mount part and the second mount part as the spacing between the rolls reduces.

In this case it is advantageous if the device for reducing the spacing between the rolls is arranged to the side of the roll arrangement, that is to say transverse to the longitudinal axes next to the rolls that form the clamping portion, since the expansion of the apparatus in the longitudinal direction is not enlarged thereby. The apparatus according to the invention can thus be handled in an improved manner. In the above-cited prior art the elements that are used to reduce a diameter are arranged before or after the clamping portion for the implant in the direction of the longitudinal axis.

In a further preferred exemplary embodiment at least one roll of the roll arrangement has a lateral surface that contains polytetrafluoroethylene (PTFE), namely, Teflon. The friction between the respective rolls and the implant is this reduced and the risk of damage to the implant is therefore further reduced. All rolls of the roll arrangement preferably have a Teflon coating on their lateral surface.

It is also preferred if the apparatus is formed rotatably about the implant or rotatably relative thereto, in particular if the apparatus is formed rotatably about the implant. The spacing may also be changed automatically.

In particular for the crimping of implants having a main structure that contains a self-expanding material, it is advantageous if the apparatus according to the invention can be introduced into a coolant. This coolant cools the implant to a temperature below the transition temperature. Since the apparatus according to the invention is relatively small, merely a relatively small container containing the coolant has to be provided, into which the apparatus according to the invention can be immersed. The size of the coolant container should be selected in this case such that the apparatus according to the invention fits therein. The container should also be sterilizable.

The above object is also achieved by a simple method for crimping an implant having the features herein.

The following steps in particular are carried out in this instance. The implant initially provided in the expanded state is first arranged over at least part of its length in an opening formed between at least three rolls of a roll arrangement. This means, for example, that one of these three rolls is initially moved relatively to the at least two other rolls in such a way that the opening provided therebetween is much greater than the outer diameter of the implant in the expanded state. The implant is then placed in the opening and the spacing between at least one roll and the at least two other rolls of the roll arrangement is reduced such that the at least three rolls initially lie closely against the outer side of the implant and thus clamp said implant therebetween. The spacing between at least one roll and the at least two further rolls is then reduced further, preferably in steps, in such a way that the implant braced between the rolls is transferred from the expanded state into the compressed state, at least over part of its length. This step described in the foregoing sentence will also be referred to hereinafter as the crimping step. The above-described advantages of the apparatus according to the invention also apply to the method according to the invention, which can additionally be carried out easily and quickly.

It is also advantageous if the entire apparatus, which carries out the method, is rotated relatively to the implant during the transfer of the implant into the compressed state so as to carry out the crimping process over the entire circumference of the implant. The apparatus is preferably rotated relatively to the implant in steps, alternately with the reduction in the spacing between the at least one roll and the at least two other rolls.

In particular for the crimping of implants containing self-expanding material such as Nitinol, it is advantageous if the apparatus for crimping is introduced into a preferably liquid coolant before the implant is transferred into the compressed state, and remains in the coolant during the crimping step.

In accordance with a preferred exemplary embodiment, so as to hold the crimped implant in the compressed state over a certain period of time until it is introduced into the body of a patient, a tube is arranged after the crimping process over the part of the length of the implant that has been transferred into the compressed state.

For example, the implant is therefore initially slid into the apparatus over part of its length, for example a proximal end, and the other end is held securely by the fingers. The spacing between the rolls is then reduced to such an extent that the implant is clamped between the rolls. The tool, including the implant, is then immersed in the coolant and is cooled to 0° C. for example so that the material of the implant comprising Nitinol for example no longer exerts any forces. The apparatus according to the invention is then rotated and the spacing is between the rolls is then reduced smoothly, step-by-step (for example the apparatus performs a complete revolution and then the roll spacing is reduced by 5 mm, then a further complete revolution of the apparatus about the clamped and securely held implant is implemented and a reduction in the roll spacing is then again implemented, etc.), until the appropriate part of the implant is crimped. The apparatus is then opened, that is to say the roll spacing is increased, and the outer tube is slid over the crimped part of the prosthesis. The crimped part of the implant fed in the outer tube is then held securely and the remaining part of the implant (that is to say the as yet uncrimped part) is transferred to a smaller diameter, similarly to the above method.

Further objectives, features, advantages and possible applications of the invention will emerge from the following description of exemplary embodiments of the invention on the basis of the drawings. All described and/or graphically illustrated features form part of the subject of the present invention, both in isolation and in any combination, irrespective of their summary in the individual claims and irrespective of the references of the claims to other claims.

### DESCRIPTION OF THE DRAWINGS

In the drawings:

FIG. 1 shows a schematic side view of a first exemplary embodiment of an apparatus according to the invention with an implant to be crimped,

FIG. 2 shows a schematic side view of a second exemplary embodiment of an apparatus according to the invention,

FIG. 3 shows a schematic cross-sectional view of a third exemplary embodiment of an apparatus according to the invention, and

FIGS. 4 and 5 show schematic cross-sectional views of the arrangement of the rolls and of the implant in exemplary embodiments 1 to 3.

### DETAILED DESCRIPTION

The figures show three exemplary embodiments in schematic and simplified form and in particular illustrate the details that are important to understand the invention. Details that are insignificant for the invention have sometimes been omitted. Furthermore, in conjunction with the present invention, the term “distal end” means the end of the implant that points away from the treating doctor as the implant is introduced into the body, whilst the “proximal end” points toward the person operating a catheter for example.

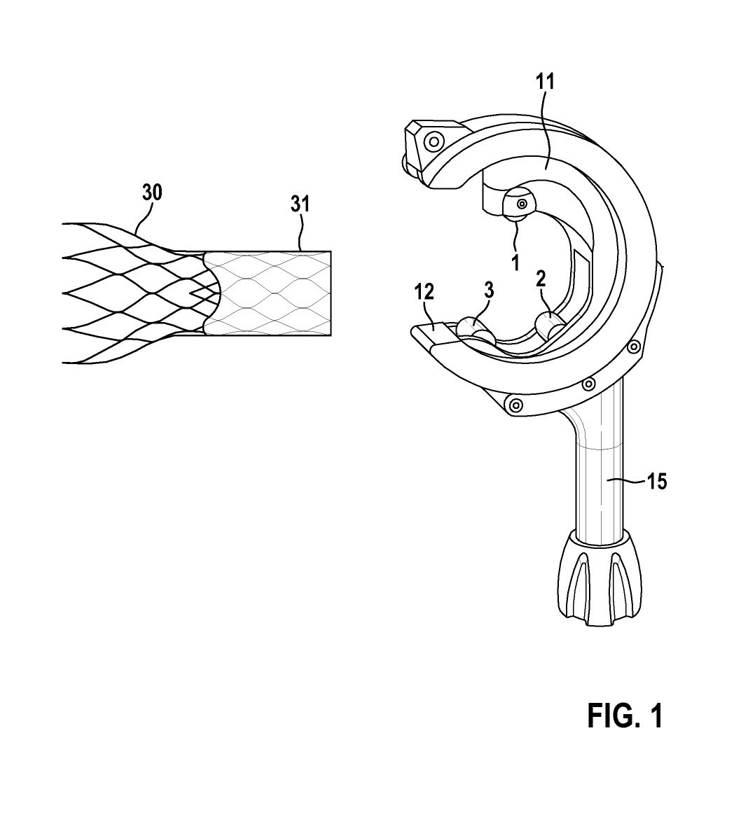

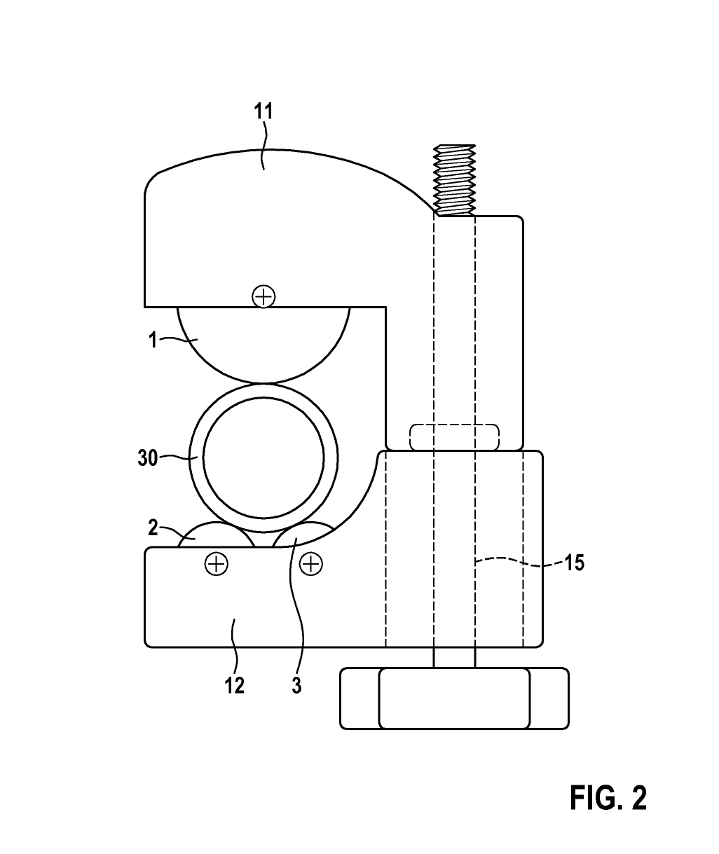

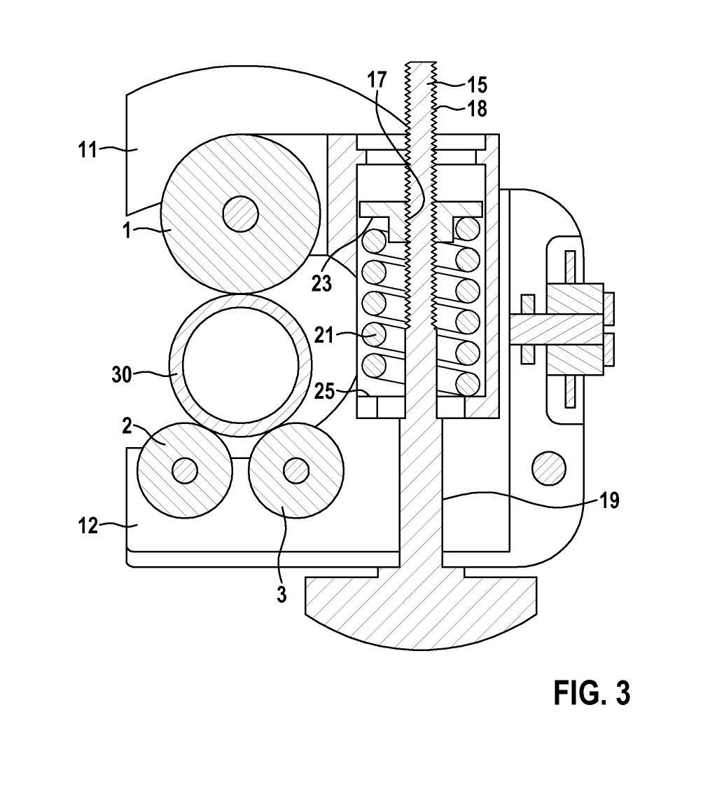

The exemplary embodiments of the apparatus according to the invention illustrated in FIGS. 1 to 3 basically differ merely in terms of the outer shaping. The fundamental technical design responsible for the crimping is identical for all three exemplary embodiments, and the apparatus will therefore be explained hereinafter in particular with reference to FIG. 3, in which the mechanics are shown most clearly. The corresponding components of the first and second exemplary embodiment are provided with the same reference signs with regard to FIG. 3.

The apparatus according to the invention has a roll arrangement comprising an upper roll 1 of slightly larger diameter and two lower rolls 2 and 3 of slightly smaller diameter. The first roll 1 is fastened rotatably to an upper, first part 11 of a C-shaped mount. The rotatable second roll 2 and the third roll 3 are arranged on a second part 12 of a C-shaped mount, spaced by a distance greater than the sum of their radii. The first part 11 of the mount is in this case arranged displaceably along a spindle 15 relative to the second part 12 of the mount in a direction transverse to the longitudinal axes of the rolls 1, 2, 3. The first part 11 of the mount has an inner thread at a through-opening 17, said inner thread cooperating with an outer thread 18 of the spindle 15 and engaging therein. The spindle 15 is mounted rotatably in the second part 12 of the mount in a through-opening 19. A compression spring 21 is also provided, which is formed as a spiral spring for example and is supported between an annular support face 23 of the first part 11 of the mount and an annular support face 25 of the second part of the mount. The compression spring 21, which receives the spindle 15 in its through-opening, is biased to press the first part 11 and the second part 12 of the mount away from one another.

By turning the spindle 15 about its longitudinal axis, the inner thread of the opening 17 of the first part 11 of the support is moved along the outer thread 18 of the spindle 15, more specifically upwardly with assistance by the force of the compression spring 21 or downwardly against the force of the compression spring 21, in accordance with the illustration in FIG. 3. The spacing D1 shown in FIG. 5 between the first roll 1 and the second roll 2 and the spacing D2 between the first roll 1 and the third roll 3 is thus increased or reduced respectively. The spacing D3 between the second roll 2 and the third roll 3 is not changed hereby in this example.

In a preferred exemplary embodiment the lateral surfaces of the rolls 1, 2 and 3 have a friction-reducing coating, for example a polytetrafluroethylene (PTFE), namely, Teflon coating, to reduce the friction between the implant and the rolls.

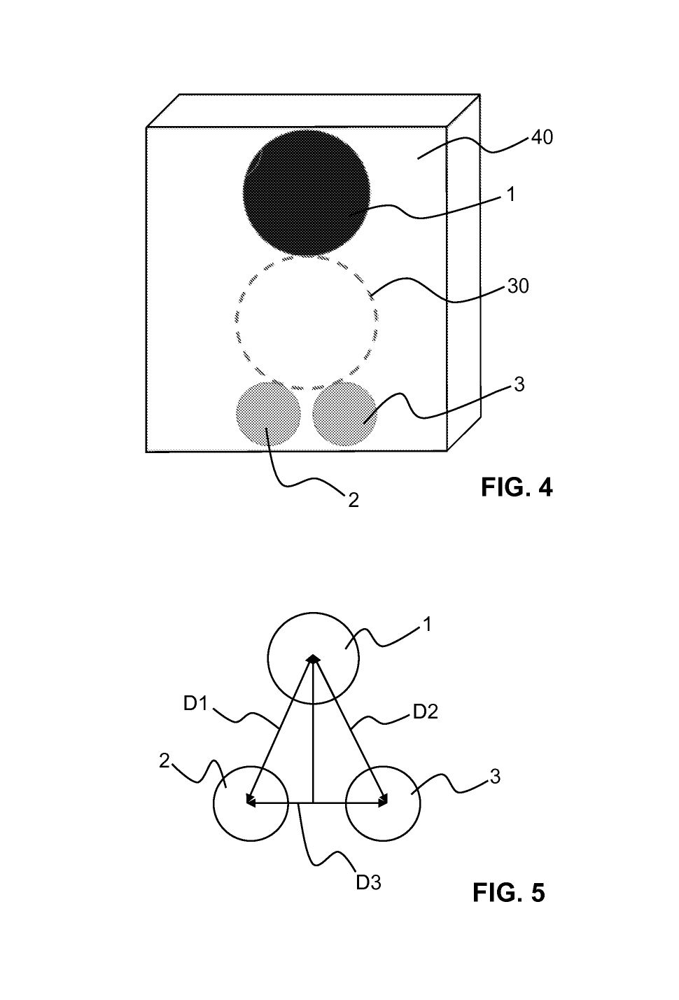

So as to crimp an implant, for example a heart valve stent, which is provided in the expanded state, the apparatus according to the invention is first opened completely. This means that the spindle 15 is rotated until the maximum spacing between the rolls 1, 2 and 3 is achieved. If necessary, the apparatus is then cooled in the coolant 40, for example in cold water having a temperature of 0° C. (see FIG. 4). The cooling step may optionally also take place before the apparatus is opened.

The implant 30 is then placed between the rolls 1, 2 and 3, preferably via its proximal end arranged opposite the distal end 31, in such a way that the longitudinal axis of the implant 30 runs approximately parallel to the longitudinal axes of the rolls 1, 2 and 3, and is clamped between the rolls 1, 2, 3. The distal end 31 of the implant, which is to lie outside the crimping pliers, is held securely by hand. It cannot be rotated. The apparatus is then rotated about the implant 30, for example by one revolution. The spindle 15 is then rotated in such a way that the spacing between the roll 1 and the rolls 2 and 3 is reduced in steps, for example by 5 mm in each case. The apparatus is then again rotated by one revolution about the implant 30. The spacing between the rolls 1, 2 and 3 is then reduced further, and a further rotation of the apparatus follows, and so on. The implant is thus preferably is crimped initially at its proximal end. In a preferred exemplary embodiment this crimping process can be carried out in a coolant 40 arranged in a suitable container (see FIG. 4), for example in cold water having a temperature of 0° C.

Once the first crimping process is complete, the spacing between the rolls 1, 2 and 3 is increased slightly by means of the spindle 15, such that a tube can be slid over the crimped proximal portion of the implant so as to fix the compressed state. The proximal end of the implant, which is already arranged beneath the outer tube of the catheter, is then held securely and the remaining portion of the implant (the distal end 31 in the present example) is crimped and the tube is slid over said portion. The crimped implant 30 fed in the tube is then removed from the apparatus and the crimping process is concluded.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

1, 2, 3 roll
11 first part of a C-shaped mount
12 second part of a C-shaped mount
15 spindle
17, 19 through-opening
18 outer thread
21 compression spring
23, 25 annular support face
30 implant
31 distal end of the implant 30
40 coolant
D1 spacing between first roll 1 and second roll 2
D2 spacing between first roll 1 and third roll 3
D3 spacing between second roll 2 and third roll 3

What is claimed is:

1. An apparatus for crimping an implant, in particular an intraluminal endoprosthesis, which can adopt either a compressed state or an expanded state, at least over part of its length, the apparatus comprising a roll arrangement comprising at least three rolls for clamping the implant therebetween, wherein spacing of at least one roll of the roll arrangement relative to each of the other at least two rolls can be reduced in such a way that an implant clamped between the rolls can be transferred from the expanded state into the compressed state, at least over part of its length.

2. The apparatus as claimed in claim 1, characterized in that each roll of the roll arrangement is fastened rotatably about its longitudinal axis in a C-shaped mount, wherein the longitudinal axes of the rolls optionally run parallel to one another and optionally further run parallel to the longitudinal axis of the implant to be crimped, when the implant is clamped between the rolls.

3. The apparatus as claimed in claim 1, characterized in that a device for reducing the spacing of the rolls relative to one another is arranged to a side of the roll arrangement.

4. The apparatus as claimed in claim 1, characterized in that at least one roll of the roll arrangement has a lateral surface that contains Teflon.

5. The apparatus as claimed in claim 1, characterized in that the apparatus is formed rotatably about the implant.

6. The apparatus as claimed in claim 1, characterized in that the apparatus can be introduced into a coolant.

7. A method for crimping an implant, in particular an intraluminal endoprosthesis, which can adopt either a compressed state or an expanded state, at least over part of its length, with use of an apparatus as claimed in claim 1, the method comprising the following steps:
arranging the implant initially provided in the expanded state in an opening formed between at least three rolls of a roll arrangement, wherein the implant is clamped between the at least three rolls; and
reducing the spacing between at least one roll and the at least two other rolls of the roll arrangement, optionally in steps, in such a way that the implant clamped between the rolls is transferred from the expanded state into the compressed state, at least over part of its length.

8. The method as claimed in claim 7, characterized in that the entire apparatus, which carries out the method, is rotated relatively to the implant during the transfer of the implant into the compressed state, optionally in steps alternately with the reduction in the spacing between the at least one roll and the at least two other rolls.

9. The method as claimed in claim 7, characterized in that the apparatus is introduced into a liquid coolant before the transfer of the implant into the compressed state, and remains in the coolant during the crimping step.

10. The method as claimed in claim 7, characterized in that, after the crimping process, a tube is arranged over the part of the length of the implant that has been transferred into the compressed state.
